(19) United States
(12) Patent Application Publication
(10) Pub. No.: US 2025/0115908 A1
(43) Pub. Date: Apr. 10, 2025

(54) METHOD OF INTERFERING WITH REPETITIVE RNA

(71) Applicant: The Regents of the University of California, Oakland, CA (US)

(72) Inventors: Eugene Yeo, La Jolla, CA (US); Aaron Smargon, San Diego, CA (US); Samuel Hatch, La Jolla, CA (US)

(21) Appl. No.: 18/836,203

(22) PCT Filed: Feb. 7, 2023
(86) PCT No.: PCT/US2023/062111

§ 371 Date: Aug. 6, 2024

Related U.S. Application Data
(60) Provisional application No. 63/307,402, filed on Feb. 7, 2022.

(51) Int. Cl. C12N 15/113

(52) U.S. Cl. CPC C12N 15/113; C12N 2310/11

(57) ABSTRACT

Provided herein are nuclear expressed antisense RNAs for interfering with a target repetitive RNA, the nuclear expressed antisense RNA comprising: (a) an oligonucleotide encoding an RNA-targeting guide comprising a sequence complementary to the target repetitive RNA; and (b) an oligonucleotide encoding a RNA binding protein (RBP)/ribonucleoprotein (RNP)-recruiting motif.

### CROSS-REFERENCE TO RELATED APPLICATIONS

This application claims priority to U.S. Provisional Patent Application No. 63/307,402, filed on Feb. 7, 2022. The disclosure of the prior application is considered part of the disclosure of this application, and is incorporated herein by reference in its entirety.

### FEDERALLY SPONSORED RESEARCH OR DEVELOPMENT

This invention was made with Government support under Grant No. HG004659 awarded by the National Institutes of Health. The Government has certain rights in the invention.

### SEQUENCE LISTING

This application contains a Sequence Listing that has been submitted electronically as an ASCII text file named Sequence Listing. The ASCII text file, created on Feb. 7, 2023, is 45,138 bytes in size. The material in the ASCII text file is hereby incorporated by reference in its entirety.

### BACKGROUND

Expansions of simple sequence repeats in RNA are responsible for dozens of diseases primarily affecting the nervous system. These diseases include myotonic dystrophy (DM1 and DM2), the most common cause of amyotrophic lateral sclerosis and frontotemporal dementia (C9ORF72 repeat expansion), and Huntington disease, among many others. Common to these diseases is the accumulation of repeat RNA in nuclear foci, thought to be mediated by sequence-specific phase transitions. These RNA foci produce cellular toxicity by sequestering RNA splicing factor RNA-binding proteins (RBPs) and through toxic protein translation in the cytoplasm, either by generating aggregation-prone translated proteins (i.e., polyglutamine peptides) or through a process known as repeat-associated nonmethionine (RAN) translation. Simple sequence repeats contributing to repeat expansion diseases include: CAG, CGG, CTG, GAA, GCC, GCG, CCTG, ATTCT, TGGAA, GGCCTG, GGGGCC, and CCCCGCCCCGCG.

Previous methods to interfere with repetitive RNA in cells or preclinical models and thus treat the underlying diseases include RNase H-mediated antisense oligo (ASO) degradation, RNA-targeting CRISPR, and repeat-targeting small molecule drugs. These methods have underlying drawbacks in that RNA interference generally occurs in the cytoplasm, and thus cannot generally be applied to treat nuclear expressed RNA in these repeat expansion diseases.

### SUMMARY

Provided herein are nuclear expressed antisense RNAs for interfering with a target repetitive RNA, the nuclear expressed antisense RNA comprising: (a) an oligonucleotide encoding an RNA-targeting guide comprising a sequence complementary to the target repetitive RNA; and (b) an oligonucleotide encoding a RNA binding protein (RBP)/ribonucleoprotein (RNP)-recruiting motif.

In some embodiments, the RBP/RNP-recruiting motif is at the 3′ end of the RNA targeting guide. In some embodiments, the RBP/RNP-recruiting motif is at the 5′ end of the RNA targeting guide.

In some embodiments, the nuclear expressed antisense RNA binds to the target repetitive RNA. In some embodiments, the target repetitive RNA comprises CUG repeats, CAG repeats, or G4C2 repeats. In some embodiments, the target repetitive RNA comprises a plurality of CUG repeats. In some embodiments, the target repetitive RNA comprises a plurality of CAG repeats.

In some embodiments, the RNA-targeting guide comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14. In some embodiments, the RNA-targeting guide comprises SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

In some embodiments, the RNA-targeting guide comprises a mutation within the sequence complementary to the target repetitive RNA. In some embodiments, the mutation comprises one or more mutation(s), and wherein the one or more mutation(s) comprise(s) a mismatch, deletion, insertion mutation, or any combination thereof.

In some embodiments, the RBP/RNP-recruiting motif recruits an endogenous mammalian RBP/RNP. In some embodiments, the endogenous mammalian RBP/RNP comprises a U snRNP component, a hnRNP component, a ribosome, a telomerase, a vault ribonucleoprotein, or an RNase P. In some embodiments, the RBP/RNP-recruiting motif comprises a U1 snRNA, a U7 smOPT snRNA, or a U7 snRNA backbone. In some embodiments, the RBP/RNP-recruiting motif comprises a U1 snRNA, wherein the U1 snRNA comprises SEQ ID NO: 29, SEQ ID NO: 30, SEQ ID NO: 31, or SEQ ID NO: 32.

In some embodiments, the nuclear expressed antisense RNA is delivered into a cell. In some embodiments, the cell is a mammalian cell. In some embodiments, the nuclear expressed antisense RNA is delivered by transfection, transduction, mechanical delivery, lipid, or transporter protein. In some embodiments, the nuclear expressed antisense RNA is delivered by a transfection reagent. In some embodiments, the nuclear expressed antisense RNA is comprised in a vector. In some embodiments, the vector is a viral vector. In some embodiments, the viral vector is an adeno-associated viral vector (AAV), lentiviral vector, or an adenoviral vector. In some embodiments, the nuclear expressed antisense RNA further comprises a Polymerase II promoter or a Polymerase III promoter sequence. In some embodiments, the nuclear expressed antisense RNA comprises a U1, U6, or U7 promoter.

Also provided herein are vectors comprising a nucleic acid encoding a nuclear expressed antisense RNA comprising (i) an RNA-targeting guide comprising a sequence complementary to a target repetitive RNA, and (ii) a RBP/RNP-recruiting motif. In some embodiments, the RBP/RNP-recruiting motif is at the 3′ end of the RNA-targeting guide. In some embodiments, the RBP/RNP-recruiting motif is at the 5′ end of the RNA-targeting guide.

In some embodiments, the RNA-targeting guide binds to the target repetitive RNA. In some embodiments, the target repetitive RNA comprises CUG repeats, CAG repeats, or G4C2 repeats. In some embodiments, the target repetitive RNA comprises a plurality of CUG repeats. In some embodiments, the target repetitive RNA comprises a plurality of CAG repeats.

In some embodiments, the RNA-targeting guide comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14. In some embodiments, the RNA-targeting guide comprises SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

In some embodiments, the RNA-targeting guide comprises a mutation within the sequence complementary to the target repetitive RNA. In some embodiments, the mutation comprises a mismatch, deletion, or insertion mutation.

In some embodiments, the RBP/RNP-recruiting motif recruits an endogenous mammalian RBP/RNP. In some embodiments, the endogenous mammalian RBP/RNP comprises a U snRNP component, a hnRNP component, a ribosome, a telomerase, a vault ribonucleoprotein, or an RNase P. In some embodiments, the RBP/RNP-recruiting motif comprises a U1 snRNA, a U7 smOPT snRNA, or a U7 snRNA backbone. In some embodiments, the RBP/RNP-recruiting motif comprises a U1 snRNA, wherein the U1 snRNA comprises SEQ ID NO: 29, SEQ ID NO: 30, SEQ ID NO: 31, or SEQ ID NO: 32.

In some embodiments, the vector is delivered into a cell. In some embodiments, the cell is a mammalian cell. In some embodiments, the vector is delivered by transfection, transduction, mechanical delivery, lipid, or transporter protein. In some embodiments, the vector is delivered by a transfection reagent. In some embodiments, the vector is a viral vector. In some embodiments, the viral vector is an adeno-associated viral vector (AAV), lentiviral vector, or an adenoviral vector. In some embodiments, the vector further comprises a Polymerase II promoter or a Polymerase III promoter sequence. In some embodiments, the vector comprises a U1, U6, or U7 promoter.

Also provided herein are cells comprising any one of the nuclear expressed antisense RNAs described herein or any one of the vectors described herein.

Also provided herein are pharmaceutical compositions comprising any one of the cells described herein and a pharmaceutically acceptable carrier.

Also provided herein are methods of treating a disease associated with expansions of a target repetitive RNA in a subject, the method comprising administering to the subject a therapeutically effective amount of any one of the pharmaceutical compositions described herein. In some embodiments, the disease is myotonic dystrophy, amyotrophic lateral sclerosis, frontotemporal dementia, Huntington disease, Friedreich ataxia, oculopharyngeal muscular dystrophy, spinocerebellar ataxia, or spinal and bulbar muscular atrophy.

Also provided herein are methods of preventing nuclear accumulation and/or protein translation of a target repetitive RNA in a cell, the method comprising delivering any one of the nuclear expressed antisense RNAs described herein or any one of the vectors described herein. In some embodiments, the cell is a mammalian cell. In some embodiments, the cell is a human cell.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1A shows an exemplary expression vector construct comprising a CUGx960 plasmid (DT960).

FIGS. 1B-1D shows results from CUG DMPK reporter knockdown in HEK293T cells with a U1 snRNA backbone (FIG. 1B), U7 smOPT snRNA backbone (FIG. 1C), and U7 snRNA (FIG. 1D).

FIG. 2A shows an exemplary expression vector construct comprising a CAGx74 plasmid (HTT).

FIG. 2B shows results from CAG HTT reporter knockdown in HEK293T cells with a U1 snRNA backbone.

FIG. 3A shows an exemplary expression vector construct comprising a CAGx57 plasmid (ATXN2).

FIG. 3B shows results from CAG ATXN22 reporter knockdown in HEK293T cells with a U1 snRNA backbone.

FIG. 4A shows an exemplary expression vector construct comprising a G4C2x66 plasmid (C9Orf72).

FIG. 4B shows results from G4C2 reporter knockdown in HEK293T cells with a U1 snRNA backbone.

FIG. 5 shows results from a tethering luciferase assay in HEK293T cells, wherein results show most RBP proteins result in knockdown of the repetitive RNA.

FIG. 6A shows exemplary expression vector constructs comprising (i) a CAGx57 plasmid and (ii) a poly(A) signal (upper) or a histone mRNA 3′ processing signal (lower).

FIG. 6B shows results from CAG reporter knockdown in HEK293T cells with a U1 snRNA backbone or a U7 smOPT snRNA.

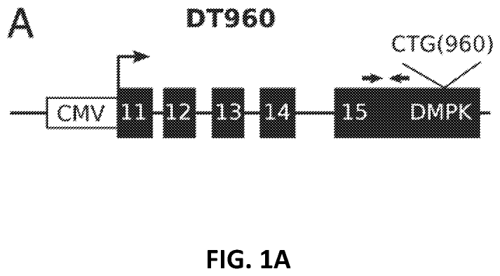

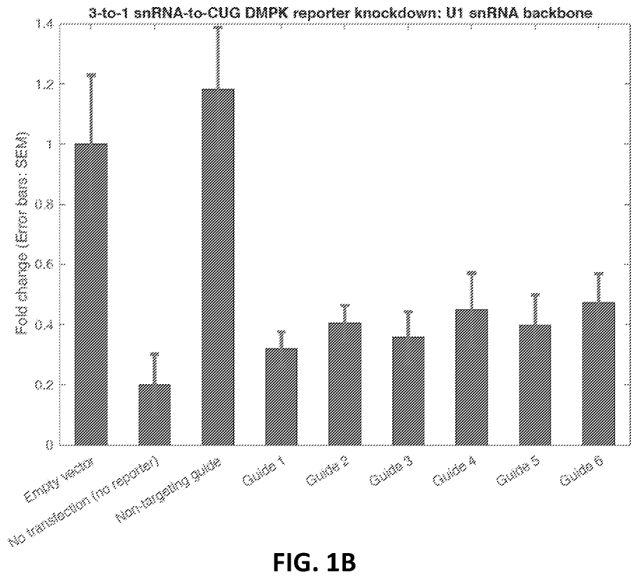

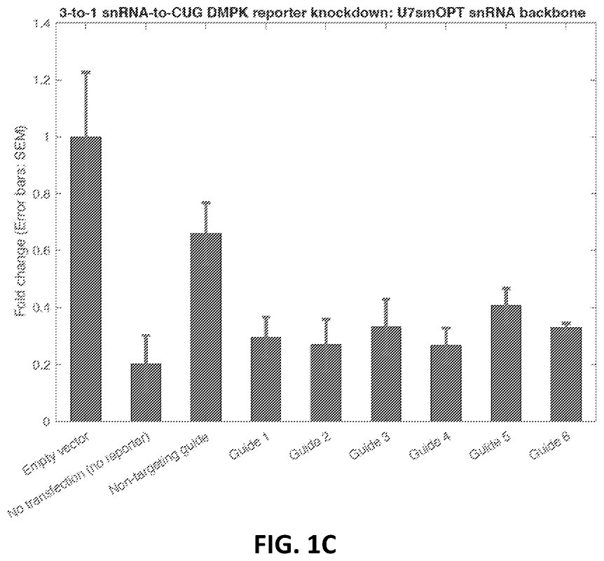

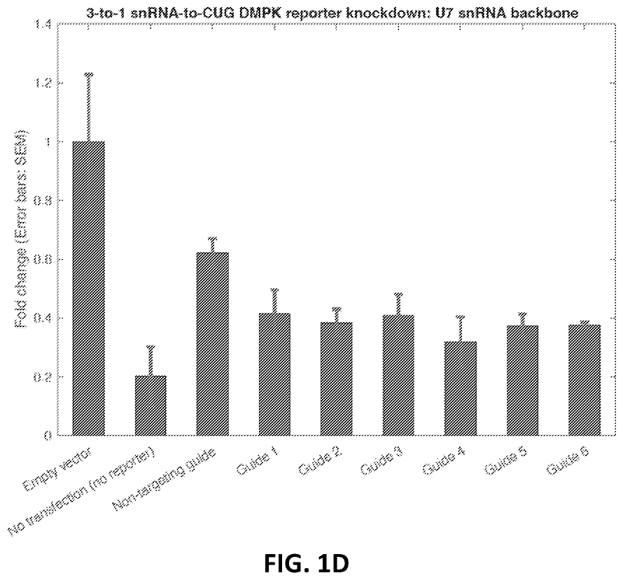

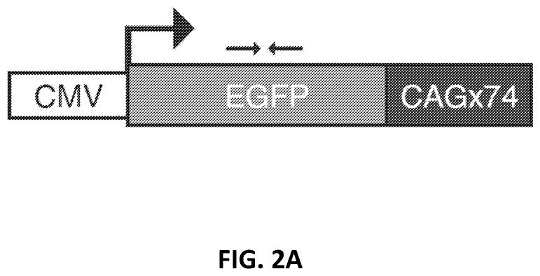

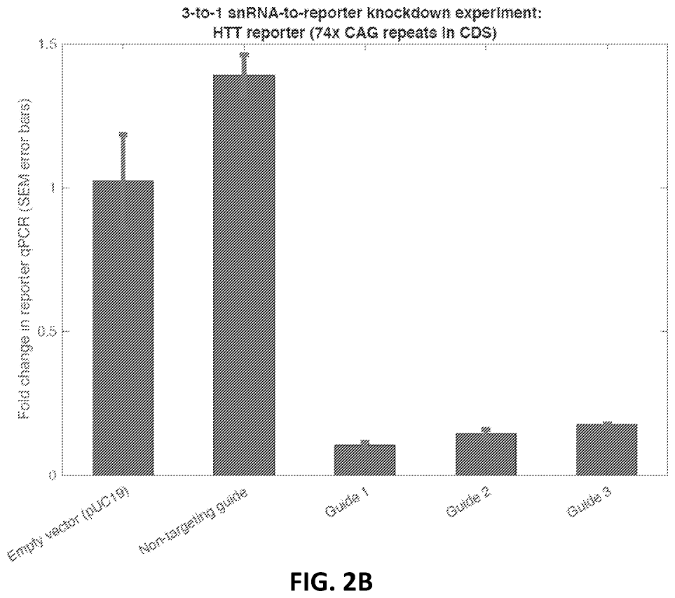

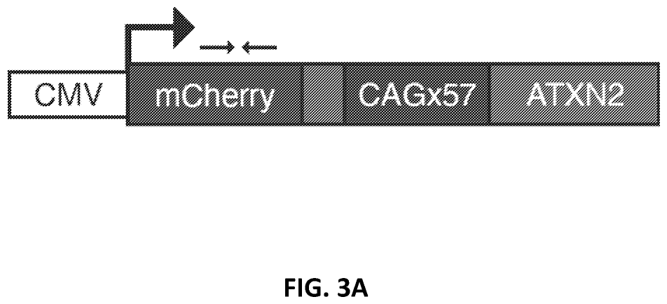

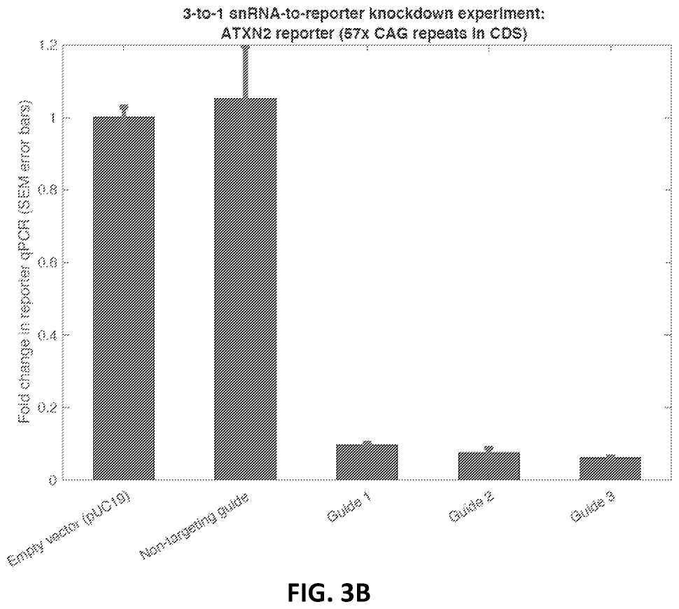

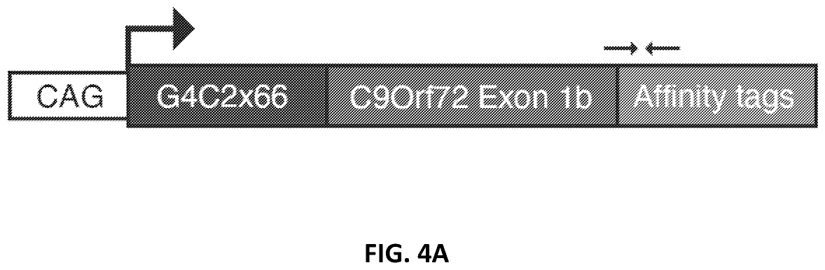

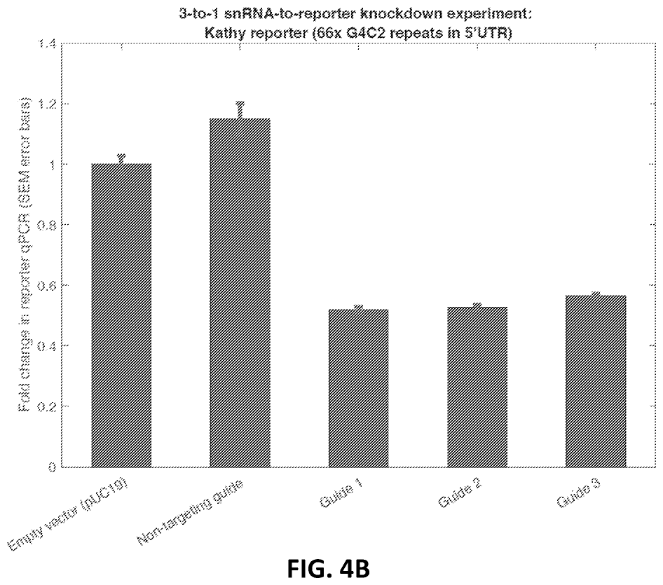

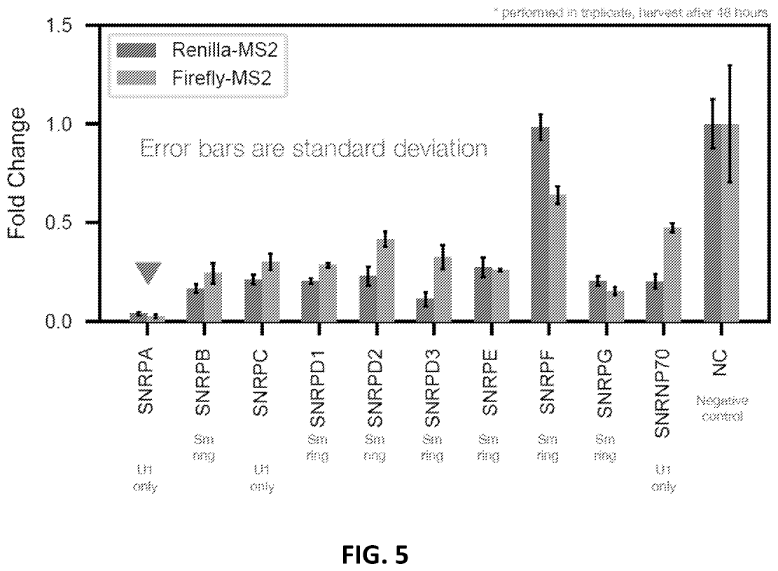

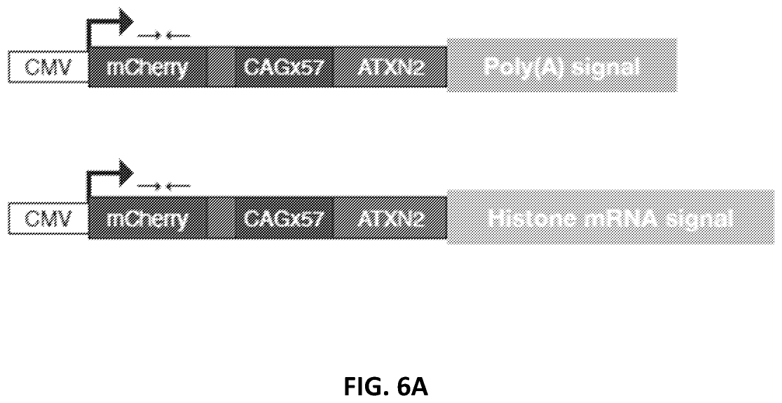

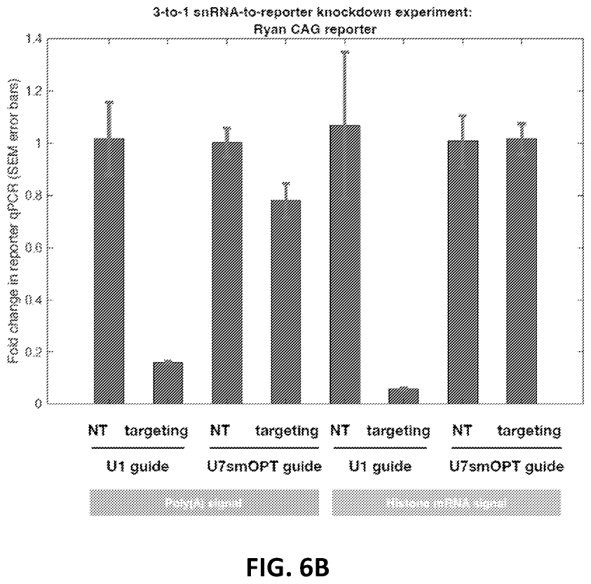

### DETAILED DESCRIPTION

The present disclosure describes nuclear expressed antisense RNAs that interfere with repetitive RNA by binding to repetitive RNA, thereby preventing the nuclear accumulation and/or protein translation of cellular RNA repeat transcripts. In some embodiments, the nuclear expressed antisense RNA comprises (i) an RNA-targeting guide comprising a sequence complementary to a target repetitive RNA, and (ii) a RBP/RNP-recruiting motif.

Various non-limiting aspects of these methods are described herein, and can be used in any combination without limitation. Additional aspects of various components of methods for regulating gene expression are known in the art.

It must be noted that, as used in the specification and the appended claims, the singular forms “a,” “an” and “the” include plural referents unless the context clearly dictates otherwise.

As used herein, the term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%,20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, a “cell” can refer to either a prokaryotic or eukaryotic cell, optionally obtained from a subject or a commercially available source.

As used herein, “delivering”, “gene delivery”, “gene transfer”, “transducing” can refer to the introduction of an exogenous polynucleotide into a host cell, irrespective of the method used for the introduction. Such methods include a variety of well-known techniques such as vector-mediated gene transfer (e.g., viral infection/transfection, or various other protein-based or lipid-based gene delivery complexes) as well as techniques facilitating the delivery of “naked” polynucleotides (e.g., electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

In some embodiments, a polynucleotide can be inserted into a host cell by a gene delivery molecule. Examples of gene delivery molecules can include, but are not limited to, liposomes, micelle biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

As used herein, the term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “exogenous” refers to any material introduced from or originating from outside a cell, a tissue or an organism that is not produced by or does not originate from the same cell, tissue, or organism in which it is being introduced.

As used herein, the term “expression” refers to the process by which polynucleotides are transcribed into mRNA and/or the process by which the transcribed mRNA is subsequently translated into peptides, polypeptides, or proteins. In some embodiments, if the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, “nucleic acid” is used to include any compound and/or substance that comprise a polymer of nucleotides. In some embodiments, a polymer of nucleotides are referred to as polynucleotides. Exemplary nucleic acids or polynucleotides can include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A deoxyribonucleic acid (DNA) can have one or more bases selected from the group consisting of adenine (A), thymine (T), cytosine (C), or guanine (G), and a ribonucleic acid (RNA) can have one or more bases selected from the group consisting of uracil (U), adenine (A), cytosine (C), or guanine (G).

In some embodiments, the term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the isolated nucleic acids described herein, the isolated nucleic acid is DNA. In some embodiments of any of the isolated nucleic acids described herein, the isolated nucleic acid is RNA.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., arginine, lysine and histidine), acidic side chains (e.g., aspartic acid and glutamic acid), uncharged polar side chains (e.g., asparagine, cysteine, glutamine, glycine, serine, threonine, tyrosine, and tryptophan), nonpolar side chains (e.g., alanine, isoleucine, leucine, methionine, phenylalanine, proline, and valine), beta-branched side chains (e.g., isoleucine, threonine, and valine), and aromatic side chains (e.g., histidine, phenylalanine, tryptophan, and tyrosine), and aromatic side chains (e.g., histidine, phenylalanine, tryptophan, and tyrosine).

As used herein, the term “nucleotides” and “nt” are used interchangeably herein to generally refer to biological molecules that comprise nucleic acids. Nucleotides can have moieties that contain the known purine and pyrimidine bases. Nucleotides may have other heterocyclic bases that have been modified. Such modifications include, e.g., methylated purines or pyrimidines, acylated purines or pyrimidines, alkylated riboses, or other heterocycles. In some embodiments, nucleic acid modifications can also include a blocking modification comprising a 3′ end modification (e.g., a 3′ dideoxy C (3′ddC), 3′ddG, 3′ddA, 3′ddT, 3′ inverted dT, 3′ C3 spacer, 3′ amino, 3′ biotinylation, or 3′ phosphorylation). The terms “polynucleotides,” “nucleic acid,” and “oligonucleotides” can be used interchangeably, and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise non-naturally occurring sequences. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, the term “plurality” can refer to a state of having a plural (e.g., more than one) number of different types of things (e.g., a cell, a genomic sequence, a subject, a system, or a protein). In some embodiments, a plurality of genomic sequences can be more than one genomic sequence wherein each genomic sequence is different from each other.

As used herein, the term “recombinant” refers to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

### A. Repetitive RNA

As used herein, “repetitive RNA” or “repetitive sequence” can refer to short or long patterns of nucleic acids (e.g., DNA or RNA) that occur in multiple copies throughout the genome. In some embodiments, these repeated sequences are necessary for maintaining important genome structures such as telomeres or centromeres. In some embodiments, repeated sequences can be important for cellular functioning and genome maintenance, while other repetitive sequences can be harmful. For example, many repetitive RNA sequences have been linked to human diseases such as Huntington's disease and Friedreich's ataxia.

In some embodiments, repetitive RNA sequences are associated with diseases. Specifically, tandem repeat sequences, underlie several human disease conditions that can include, but are not limited to, Huntington's disease, fragile X syndrome, several spinocerebellar ataxias, myotonic dystrophy and Friedreich's ataxia. In some embodiments, repetitive RNA sequences can include trinucleotide repeat expansions, wherein the trinucleotide repeat expansions may occur during DNA replication or during DNA repair synthesis. For example, genes containing pathogenic CAG repeats often encode proteins that have a role in the DNA damage response and repeat expansions of these repeats may impair specific DNA repair pathways.

In some embodiments, a disease associated with expansions of a repetitive RNA can include Fragile X Syndrome and Fragile X Tremor Ataxia Syndrome (FXS/FXTAS), CAG/polyglutamine diseases, spinal and bulbar muscular atrophy (SBMA), Huntington disease (HD), spinocerebellar ataxias (SCAs), myotonic dystrophy, Friedreich ataxia, C9ORF72 frontotemporal dementia/amyotrophic lateral sclerosis (ALS), congenital neurocognitive disorders, oculopharyngeal muscular dystrophy (OPMD), adult onset neuromuscular disorder, and Unverricht-Lundborg myoclonic epilepsy (EPM1). In some embodiments, a repeat expansion of CAG can cause Huntington disease, spinal and bulbar muscular atrophy, dentatorubral-pallidoluysian atrophy, or spinocerebellar ataxias (SCAs). In some embodiments, a repeat expansion of CGG can cause fragile X, or fragile X tremor ataxia syndrome. In some embodiments, a repeat expansion of CTG can cause myotonic dystrophy type 1, Huntington disease-like 2, spinocerebellar ataxia type 8, or Fuchs corneal dystrophy. In some embodiments, a repeat expansion of GAA can cause Friedreich ataxia. In some embodiments, a repeat expansion of GCC can cause FRAXE mental retardation. In some embodiments, a repeat expansion of GCG can cause oculopharyngeal muscular dystrophy. In some embodiments, a repeat expansion of CCTG can cause myotonic dystrophy type 1. In some embodiments, a repeat expansion of ATTCT can cause spinocerebellar ataxia type 10. In some embodiments, a repeat expansion of TGGAA can cause spinocerebellar ataxia type 31. In some embodiments, a repeat expansion of GGCCTG can cause spinocerebellar ataxia type 36. In some embodiments, a repeat expansion of GGGGCC can cause C9ORF72 frontotemporal dementia/amyotrophic lateral sclerosis. In some embodiments, a repeat expansion of CCCCGCCCCGCG can cause EPM1 (myoclonic epilepsy).

### B. Nuclear Expressed Antisense RNA

Provided herein are nuclear expressed antisense RNA for interfering with a target repetitive RNA comprising: (a) an oligonucleotide encoding an RNA-targeting guide comprising a sequence complementary to the target repetitive RNA; and (b) an oligonucleotide encoding a RBP (RNA binding protein)/RNP (ribonucleoprotein)-recruiting motif.

As used herein, a “target repetitive RNA” can refer to a nucleic acid capable of being targeted by an antisense oligonucleotide. A “target repetitive RNA” comprises a repeat expansion sequence. In some embodiments, a repetitive RNA can include a repeat expansion of a repeat sequence that occurs 2 or more (e.g., 3 or more, 4 or more, 5 or more, 10 or more, 20 or more, 30 or more, 40 or more, 50 or more, 100 or more, 150 or more, 200 or more, 300 or more, 400 or more, 500 or more, 600 or more, 700 or more, 800 or more, 900 or more, 1000 or more, 2000 or more, 3000 or more, 4000 or more, or 5000 or more) times within a gene. In some embodiments, the repeat sequence can be 100% identical to each other. In some embodiments, the repeat sequence is not 100% identical.

In some embodiments, the repeating sequence comprises about 3 to about 12 (e.g., about 3 to about 11, about 3 to about 10, about 3 to about 9, about 3 to about 8, about 3 to about 7, about 3 to about 6, about 3 to about 5, about 3 to about 4, about 4 to about 12, about 4 to about 11, about 4 to about 10, about 4 to about 9, about 4 to about 8, about 4 to about 7, about 4 to about 6, about 4 to about 5, about 5 to about 12, about 5 to about 11, about 5 to about 10, about 5 to about 9, about 5 to about 8, about 5 to about 7, about 5 to about 6, about 6 to about 12, about 6 to about 11, about 6 to about 10, about 6 to about 9, about 6 to about 8, about 6 to about 7, about 7 to about 12, about 7 to about 11, about 7 to about 10, about 7 to about 9, about 7 to about 8, about 8 to about 12, about 8 to about 11, about 8 to about 10, about 8 to about 9, about 9 to about 12, about 9 to about 11, about 9 to about 10, about 10 to about 12, about 10 to about 11, or about 11 to about 12) nucleotides in length. In some embodiments, a repeat sequence may be repeated in tandem or multiple repeat regions, which may be near each other such as, for example, within 100 nucleobases.

In some embodiments, the repeating sequence can comprise a CAG, CGG, CTG, GAA, GCC, GCG, CCTG, ATTCT, TGGAA, GGCCTG, GGGGCC, or CCCCGCCCCGCG sequence. In some embodiments, the repeating sequence can comprise a CUG sequence. In some embodiments, the repeating sequence can comprise a CAG sequence. In some embodiments, the repeating sequence can comprise a G4C2 sequence. In some embodiments, the target repetitive RNA can comprise a CUG sequence. In some embodiments, the target repetitive RNA can comprise a CAG sequence. In some embodiments, the target repetitive RNA can comprise a G4C2 sequence. In some embodiments, the target repetitive RNA comprises CUG repeats, CAG repeats, or G4C2 repeats. In some embodiments, the target repetitive RNA comprises a plurality of CUG repeats. In some embodiments, the target repetitive RNA comprises a plurality of CAG repeats.

As used herein, an “antisense RNA” or “antisense oligonucleotide” refers to a single-stranded oligonucleotide having a nucleobase sequence that allows hybridization to a corresponding region or segment of a target nucleic acid. An oligonucleotide can be “antisense” to a target nucleic acid, meaning that it is capable of undergoing hybridization to the target nucleic acid through hydrogen bonding. In some embodiments, an antisense RNA has a nucleobase sequence that comprises the complement of the target nucleic acid to which it is targeted. A “nuclear expressed antisense RNA” can refer to an antisense RNA that is expressed in the nucleus.

In some embodiments, the antisense RNA can be about 10 to about 100 (e.g., about 10 to about 90, about 10 to about 80, about 10 to about 70, about 10 to about 60, about 10 to about 50, about 10 to about 40, about 10 to about 30, about 10 to about 20, about 20 to about 100, about 20 to about 90, about 20 to about 80, about 20 to about 70, about 20 to about 60, about 20 to about 50, about 20 to about 40, about 20 to about 30, about 30 to about 100, about 30 to about 90, about 30 to about 80, about 30 to about 70, about 30 to about 60, about 30 to about 50, about 30 to about 40, about 40 to about 100, about 40 to about 90, about 40 to about 80, about 40 to about 70, about 40 to about 60, about 40 to about 50, about 50 to about 100, about 50 to about 90, about 50 to about 80, about 50 to about 70, about 50 to about 60, about 60 to about 100, about 60 to about 90, about 60 to about 80, about 60 to about 70, about 70 to about 100, about 70 to about 90, about 70 to about 80, about 80 to about 100, about 80 to about 90, or about 90 to about 100) nucleotides in length. In some embodiments, the antisense RNA can be about 10 to about 30 nucleotides in length.

In some embodiments, the nuclear expressed antisense RNA can bind to the target repetitive RNA. In some embodiments, the nuclear expressed antisense RNA stays bound to the target repetitive RNA.

In some embodiments, the nuclear expressed antisense RNA further comprises a Polymerase II promoter sequence. In some embodiments, the nuclear expressed antisense RNA further comprises a Polymerase III promoter sequence. In some embodiments, the nuclear expressed antisense RNA further comprises a Polymerase II promoter sequence or a Polymerase III promoter sequence. In some embodiments, the nuclear expressed antisense RNA comprises a U1, U6, or U7 promoter. In some embodiments, the Polymerase II promoter comprises a U1 or U7 promoter. In some embodiments, the Polymerase III promoter comprises a U6 promoter.

As used herein, an “RNA-targeting guide” refers to an RNA sequence used to target specific RNA sequences within a gene. In some embodiments, the RNA-targeting guide can recognize a target repetitive RNA, for example, by hybridizing to the target repetitive RNA. In some embodiments, the RNA-targeting guide comprises a sequence that is complementary to the target repetitive RNA. In some embodiments, the RNA-targeting guide can include one or more modified nucleotides. In some embodiments, the RNA-targeting guide comprises a mutation within the sequence complementary to the target repetitive RNA. In some embodiments, the mutation comprises a mismatch, deletion, or insertion mutation. In some embodiments, the RNA-targeting guide comprises one or more mutation(s) within the sequence complementary to the target repetitive RNA. In some embodiments, the one or more mutation(s) comprise(s) a mismatch, deletion, insertion mutation, or any combination thereof. In some embodiments, the RNA-targeting guide comprises a plurality of mutations, wherein the plurality of mutations include a mismatch, a deletion, an insertion mutation, or any combination thereof. In some embodiments, these mutations can be introduced into the RNA-targeting guide to reduce binding affinity of the RNA-targeting guide for the targeted repetitive RNA.

In some embodiments, the RNA-targeting guide comprises a sequence targeting CUG repeats (Table 1). In some embodiments, the RNA-targeting guide comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14. In some embodiments, the RNA-targeting guide comprises a sequence targeting CAG repeats (Table 2). In some embodiments, the RNA-targeting guide comprises SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

TABLE 1

| Construct | | SEQ |
|---|---|---|
| Name | Guide sequence (5-3′) | ID NO |
| U1 25 nt NTC | GATGGAATGTCCTTAACTCTCGCAC | 1 |
| U1 15 nt CUG1 | CAGCAGCAGCAGCAG | 2 |
| U1 15 nt CUG2 | AGCAGCAGCAGCAGC | 3 |
| U1 15 nt CUG3 | GCAGCAGCAGCAGCA | 4 |
| U1 20 nt CUG1 | AGCAGCAGCAGCAGCAGCAG | 5 |
| U1 20 nt CUG2 | GCAGCAGCAGCAGCAGCAGC | 6 |
| U1 20 nt CUG3 | CAGCAGCAGCAGCAGCAGCA | 7 |
| U7smOPT 25 nt | GATGGAATGTCCTTAACTCTCGCAC | 8 |
| NTC | | |
| U7smOPT 15 nt | CAGCAGCAGCAGCAG | 9 |
| CUG1 | | |
| U7smOPT 15 nt | AGCAGCAGCAGCAGC | 10 |
| CUG2 | | |
| U7smOPT 15 nt | GCAGCAGCAGCAGCA | 11 |
| CUG3 | | |
| U7smOPT 20 nt | AGCAGCAGCAGCAGCAGCAG | 12 |
| CUG1 | | |
| U7smOPT 20 nt | GCAGCAGCAGCAGCAGCAGC | 13 |
| CUG2 | | |
| U7smOPT 20 nt | CAGCAGCAGCAGCAGCAGCA | 14 |
| CUG3 | | |

TABLE 2

| Construct | | SEQ |
|---|---|---|
| Name | Guide sequence (5-3′) | ID NO |
| U1 25 nt NTC | GATGGAATGTCCTTAACTCTCGCAC | 15 |
| U1 15 nt CAG1 | CTGCTGCTGCTGCTG | 16 |
| U1 15 nt CAG2 | TGCTGCTGCTGCTGC | 17 |
| U1 15 nt CAG3 | GCTGCTGCTGCTGCT | 18 |
| U1 20 nt CAG1 | TGCTGCTGCTGCTGCTGCTG | 19 |
| U1 20 nt CAG2 | GCTGCTGCTGCTGCTGCTGC | 20 |
| U1 20 nt CAG3 | CTGCTGCTGCTGCTGCTGCT | 21 |
| U7smOPT 25 nt | GATGGAATGTCCTTAACTCTCGCAC | 22 |
| NTC | | |
| U7smOPT 15 nt | CTGCTGCTGCTGCTG | 23 |
| CAG1 | | |
| U7smOPT 15 nt | TGCTGCTGCTGCTGC | 24 |
| CAG2 | | |
| U7smOPT 15 nt | GCTGCTGCTGCTGCT | 25 |
| CAG3 | | |
| U7smOPT 20 nt | TGCTGCTGCTGCTGCTGCTG | 26 |
| CAG1 | | |
| U7smOPT 20 nt | GCTGCTGCTGCTGCTGCTGC | 27 |
| CAG2 | | |
| U7smOPT 20 nt | CTGCTGCTGCTGCTGCTGCT | 28 |
| CAG3 | | |

As used herein, an “RBP/RNP-recruiting motif” refers to a region of a nucleic acid sequence (e.g., DNA or RNA) that has a specific structure to recruit RNA binding protein (RBP)/ribonucleoprotein (RNP) complexes. In some embodiments, the RBP/RNP-recruiting motif is at the 3′ end of the RNA targeting guide. In some embodiments, the RBP/RNP-recruiting motif is at the 5′ end of the RNA targeting guide.

In some embodiments, the RBP/RNP-recruiting motif recruits an endogenous mammalian RBP/RNP. In some embodiments, the RBP/RNP-recruiting motif comprises a U1 snRNA, a U7 smOPT snRNA, or a U7 snRNA backbone. As used herein, an “RNA backbone” refers to a phosphate backbone of an RNA strand that includes alternating sugar (deoxyribose) and phosphate groups, wherein attached to each sugar is one of four bases—adenine (A), cytosine (C), guanine (G), or thymine (T).

In some embodiments, the RBP/RNP-recruiting motif comprises a U1 snRNA. In some embodiments, the U1 snRNA comprises a U1 snRNA Sm binding motif, wherein the U1 snRNA Sm binding motif comprises SEQ ID NO: 29. In some embodiments, the U1 snRNA comprises a U1 snRNA stem loop 4, wherein the U1 snRNA stem loop 4 comprises SEQ ID NO: 30. In some embodiments, the U1 snRNA comprises a U1 snRNA stem loop 2, wherein the U1 snRNA stem loop 2 comprises SEQ ID NO: 31. In some embodiments, the U1 snRNA comprises a U1 snRNA stem loop 1, wherein the U1 snRNA stem loop 1 comprises SEQ ID NO: 32.

As used herein, an “endogenous mammalian RBP/RNP” refers to a RNA binding protein (RBP) and/or ribonucleoprotein (RNP) that form a complex. RNAs and RNA binding proteins (RBPs) can interact dynamically in ribonucleoprotein (RNP) complexes to determine posttranscriptional control of gene expression and influencing protein production. In some embodiments, the endogenous mammalian RBP/RNP comprises a U snRNP component, a hnRNP component, a ribosome, a telomerase, a vault ribonucleoprotein, or an RNase P. In some embodiments, the endogenous mammalian RBP/RNP comprises a nuclear RNA binding protein. In some embodiments, the endogenous mammalian RBP/RNP comprises SNRPA, SNRPB, SNRPC, SNRPD1, SNRPD2, SNRPD3, SNRPE, SNRPF, SNRPG, SNRNP70, hnRNP A/B, hnRNP C, hnRNP D, hnRNP F, hnRNP H, hnRNP I, hnRNP K, hnRNP L, hnRNP M, hnRNP R, or hnRNP U.

TABLE 3

| | Sequence | SEQ ID NO |
|---|---|---|
| U1 snRNA Sm | ATAATTTGTGGTAGTG | 29 |
| binding motif | | |
| U1 snRNA stem | GGGGACTGCGTTCGCGCTTTCCCCTG | 30 |
| loop 4 | | |
| U1 snRNA stem | CAGGGCGAGGCTTATCCATTGCACTCCGGATGTGCT | 31 |
| loop 2 (binds | GACCCCTG | |
| U1A/SNRPA) | | |
| U1 snRNA stem | GGGAGATACCATGATCACGAAGGTGGTTTTCCC | 32 |
| loop 1 (binds | | |
| U1-70K/ | | |
| SNRNP70) | | |

In some embodiments, the nuclear expressed antisense RNAs described herein can be delivered into a cell. In some embodiments, the cell is a mammalian cell. In some embodiments, the nuclear expressed antisense RNA is delivered by transfection (e.g., using transfectamine, cationic polymers, calcium phosphate or electroporation), transduction (e.g., using a bacteriophage or recombinant viral vector), mechanical delivery (e.g., magnetic beads), lipid (e.g., 1,2-Dioleoyl-sn-glycero-3-phosphocholine (DOPC)), or transporter protein. In some embodiments, the nuclear expressed antisense RNA is delivered by a transfection reagent.

In some embodiments, the nuclear expressed antisense RNA is comprised in a vector. In some embodiments, a vector can be an expression vector where the expression vector includes a promoter sequence operably linked to a sequence encoding a molecule (e.g., a nucleic acid encoding a nuclear expressed antisense RNA). Non-limiting examples of vectors include plasmids, transposons, cosmids, and viral derived vectors (e.g., any adenoviral derived vectors (AV) cytomegaloviral derived (CMV) vectors, simian viral derived (SV40) vectors, adeno-associated virus (AAV) vectors, lentivirus vectors, and retroviral vectors), and any Gateway® vectors. In some embodiments, a vector can include sufficient cis-acting elements for expression where other elements for expression can be supplied by the host mammalian cell or in an in vitro expression system. In some embodiments, the vector is a viral vector. In some embodiments, the viral vector is an adeno-associated viral vector (AAV), lentiviral vector, or an adenoviral vector.

Also provided herein are vectors comprising a nucleic acid encoding a nuclear expressed antisense RNA that include (i) an RNA-targeting guide comprising a sequence complementary to a target repetitive RNA, and (ii) a RBP/RNP-recruiting motif.

### C. Pharmaceutical Compositions and Therapeutic Applications

Provided herein are cells comprising any one of the nuclear expressed antisense RNAs or any one of the vectors described herein. Also provided herein are pharmaceutical compositions comprising any one of the cells described herein and a pharmaceutically acceptable carrier.

As used herein, the term “pharmaceutical composition” refers to a composition in which an active agent is formulated together with one or more pharmaceutically acceptable carriers. In some embodiments, a pharmaceutical composition can include a buffer, a diluent, solubilizer, emulsifier, preservative, adjuvant, an excipient, or any combination thereof. In some embodiments, a composition, if desired, can also contain one or more additional therapeutically active substances. In some embodiments, the composition is suitable for administration to a human or animal subject. In some embodiments, the active agent is present in unit dose amount appropriate for administration in a therapeutic regimen that shows a statistically significant probability of achieving a predetermined therapeutic effect when administered to a relevant population.

Also provided herein are methods of treating a disease associated with expansions of a target repetitive RNA in a subject, the method including administering to the subject a therapeutically effective amount of any one of the pharmaceutical compositions described herein. As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

### D. Administration of the Nuclear Expressed Antisense RNA

As used herein, the term “subject” refers an organism, typically a mammal (e.g., a human). In some embodiments, a subject is suffering from a relevant disease, disorder, or condition. In some embodiments, a subject is susceptible to a disease, disorder, or condition. In some embodiments, a subject displays one or more signs or symptoms or characteristics of a disease, disorder, or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, etc. In some particular embodiments, administration may be bronchial (e.g., by bronchial instillation), buccal, dermal (which may be or comprise, for example, one or more of topical to the dermis, intradermal, transdermal, etc.), enteral, intra-arterial, intra-venous, intragastric, intramedullary, intramuscular, intranasal, intraperitoneal, intrathecal, intravenous, intraventricular, intracisternal, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

As used herein, the term “treating” means a reduction in the number, frequency, severity, or duration of one or more (e.g., two, three, four, five, or six) symptoms of a disease or disorder in a subject (e.g., any of the subjects described herein), and/or results in a decrease in the development and/or worsening of one or more symptoms of a disease or disorder in a subject.

In some embodiments, the administration of the nuclear expressed antisense RNA to a subject can result in the nuclear expressed antisense RNA being constitutively expressed in the cells of the subject. In some embodiments, the administration of the nuclear expressed antisense RNA involves a single dose. In some embodiments, the administration of the nuclear expressed antisense RNA does not require repeated administration. Also provided herein are methods of delivering any one of the nuclear expressed antisense RNAs or any one of the vectors described herein for the prevention of nuclear accumulation and/or protein translation of a target repetitive RNA in a cell.

In some embodiments, the nuclear expressed antisense RNA comprises a gene promoter, a gene, and a gene terminator, wherein the antisense RNA comprises a small genetic payload that is less than 1000 base pairs (e.g., less than 900 base pairs, less than 800 base pairs, less than 700 base pairs, less than 600 base pairs, less than 500 base pairs, less than 400 base pairs, less than 300 base pairs, less than 200 base pairs, or less than 100 base pairs). In some embodiments, the nuclear expressed antisense RNA cannot be translationally silenced after being administrated to the subject. In some embodiments, the administration of the nuclear expressed antisense RNA presents no potential immunogenicity from an exogenous protein in the cells of the subject. In some embodiments, the administration of the nuclear expressed antisense RNA presents less toxic off-target effects in the cells of the subject. In some embodiments, the nuclear expressed antisense RNA can be engineered to target different repeat expansion sequences. In some embodiments, the administration of the nuclear expressed antisense RNA can be used to treat multiple repeat expansion diseases.

In some embodiments, the nuclear expressed antisense RNA is administered to a subject having a disease associated with expansions of the target repetitive RNA. In some embodiments, the nuclear expressed antisense RNA is administered to treat a disease or condition. In some embodiments, the disease or condition is associated with expansions of target repetitive RNA, including, but not limited to, myotonic dystrophy, amyotrophic lateral sclerosis, frontotemporal dementia, Huntington disease, Friedreich ataxia, oculopharyngeal muscular dystrophy, spinocerebellar ataxia, or spinal and bulbar muscular atrophy. In some embodiments, the disease associated with expansions of a repetitive RNA can include Fragile X Syndrome and Fragile X Tremor Ataxia Syndrome (FXS/FXTAS), CAG/polyglutamine diseases, spinal and bulbar muscular atrophy (SBMA), Huntington disease (HD), spinocerebellar ataxias (SCAs), myotonic dystrophy, Friedreich ataxia, C9ORF72 frontotemporal dementia/amyotrophic lateral sclerosis (ALS), congenital neurocognitive disorders, oculopharyngeal muscular dystrophy (OPMD), adult onset neuromuscular disorder, and Unverricht-Lundborg myoclonic epilepsy (EPM1).

In some embodiments, the nuclear expressed antisense RNA is administered to a subject having a repeat expansion of CAG. In some embodiments, the nuclear expressed antisense RNA is administered to a repeat expansion of CAG in order to treat a disease or condition associated with the CAG repeat expansion. In some embodiments, repeat expansions of CAG are associated with Huntington disease, spinal and bulbar muscular atrophy, dentatorubral-pallidoluysian atrophy, or spinocerebellar ataxias (SCAs). In some embodiments, a repeat expansion of CGG can cause fragile X, or fragile X tremor ataxia syndrome. In some embodiments, a repeat expansion of CTG can cause myotonic dystrophy type 1, Huntington disease-like 2, spinocerebellar ataxia type 8, or Fuchs corneal dystrophy. In some embodiments, a repeat expansion of GAA can cause Friedreich ataxia. In some embodiments, a repeat expansion of GCC can cause FRAXE mental retardation. In some embodiments, a repeat expansion of GCG can cause oculopharyngeal muscular dystrophy. In some embodiments, a repeat expansion of CCTG can cause myotonic dystrophy type 1. In some embodiments, a repeat expansion of ATTCT can cause spinocerebellar ataxia type 10. In some embodiments, a repeat expansion of TGGAA can cause spinocerebellar ataxia type 31. In some embodiments, a repeat expansion of GGCCTG can cause spinocerebellar ataxia type 36. In some embodiments, a repeat expansion of GGGGCC can cause C9ORF72 frontotemporal dementia/amyotrophic lateral sclerosis. In some embodiments, a repeat expansion of CCCCGCCCCGCG can cause EPM1 (myoclonic epilepsy). The nuclear expressed antisense RNAs of the disclosure can be constructed and administered to a subject having any of the conditions identified herein for the treatment of that condition.

In some embodiments, the nuclear expressed antisense RNAs are administered to cells in vitro. In some embodiments, the cell is a mammalian cell. In some embodiments, the cell is a human cell. In some embodiments, the nuclear expressed antisense RNAs are administered to cells in vivo, via gene transfection.

As used herein, the term “transduced”, “transfected”, or “transformed” refers to a process by which exogenous nucleic acid is introduced or transferred into a cell. A “transduced,” “transfected,” or “transformed” mammalian cell is one that has been transduced, transfected or transformed with exogenous nucleic acid (e.g., a gene delivery vector) that includes an exogenous nucleic acid. The transfection can occur by delivering a vector to a cell, wherein the vector encodes any of the nuclear expressed antisense RNAs described herein.

As used herein, a “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual 2^{nd} ed., Cold Spring Harbor Laboratory Press, Cold Spring Harbor, N.Y. (1989)), which is incorporated herein by reference for any purpose.

### Examples

### Basis Plasmids

U1 and U7 smOPT basis plasmids were used as distinct pUC-19 backbones into which designed/synthesized/annealed guides were inserted (Tables 4-5). Synthesized/annealed oligos were ligated into pre-BbsI restriction digested U1 or U7 smOPT basis plasmids. When cloned, guide sequences provided herein replace the “Placeholder site with 2× opposing BbsI type II RE sites” sequence.

TABLE 4

| | Sequence (5′-3′) | SEQ ID NO |
|---|---|---|
| U1 plasmid | TAACACAGGCTAAGGACCAGCTTCTTTGGGAGAGAACAGACGCAGG | 33 |
| sequence | GGCGGGAGGGAAAAAGGGAGAGGCAGACGTCACTTCCCCTTGGCGG | |
| | CTCTGGCAGCAGATTGGTCGGTTGAGTGGCAGAAAGGCAGACGGGG | |
| | ACTGGGCAAGGCACTGTCGGTGACATCACGGACAGGGCGACTTCTA | |
| | TGTAGATGAGGCAGCGCAGAGGCTGCTGCTTCGCCACTTGCTGCTTC | |
| | ACCACGAAGGAGTTCCCGTGCCCTGGGAGCGGGTTCAGGACCGCTG | |
| | ATCGGAAGTGAGAATCCCAGCTGTGTGTCAGGGCTGGAAAGGGCTC | |
| | GGGAGTGCGCGGGGCAAGTGACCGTGTGTGTAAAGAGTGAGGCGTA | |
| | TGAGGCTGTGTCGGGGCAGAGGCCCAAGATCTCAGGGTCTTCGAGA | |
| | AGACCTGCAGGGGAGATACCATGATCACGAAGGTGGTTTTCCCAGG | |
| | GCGAGGCTTATCCATTGCACTCCGGATGTGCTGACCCCTGCGATTTC | |
| | CCCAAATGTGGGAAACTCGACTGCATAATTTGTGGTAGTGGGGGACT | |
| | GCGTTCGCGCTTTCCCCTGACTTTCTGGAGTTTCAAAAGTAGACTGT | |
| | ACGCTAA | |
| U1 Promoter | TAACACAGGCTAAGGACCAGCTTCTTTGGGAGAGAACAGACGCAGG | 34 |
| | GGCGGGAGGGAAAAAGGGAGAGGCAGACGTCACTTCCCCTTGGCGG | |
| | CTCTGGCAGCAGATTGGTCGGTTGAGTGGCAGAAAGGCAGACGGGG | |
| | ACTGGGCAAGGCACTGTCGGTGACATCACGGACAGGGCGACTTCTA | |
| | TGTAGATGAGGCAGCGCAGAGGCTGCTGCTTCGCCACTTGCTGCTTC | |
| | ACCACGAAGGAGTTCCCGTGCCCTGGGAGCGGGTTCAGGACCGCTG | |
| | ATCGGAAGTGAGAATCCCAGCTGTGTGTCAGGGCTGGAAAGGGCTC | |
| | GGGAGTGCGCGGGGCAAGTGACCGTGTGTGTAAAGAGTGAGGCGTA | |
| | TGAGGCTGTGTCGGGGCAGAGGCCCAAGATCTC | |
| Placeholder | GGGTCTTCGAGAAGACCT | 35 |
| site with 2x | | |
| opposing BbsI | | |
| type II RE sites | | |
| U1 snRNA | GCAGGGGAGATACCATGATCACGAAGGTGGTTTTCCCAGGGCGAGG | 36 |
| backbone | CTTATCCATTGCACTCCGGATGTGCTGACCCCTGCGATTTCCCCAAAT | |
| | GTGGGAAACTCGACTGCATAATTTGTGGTAGTGGGGGACTGCGTTCG | |
| | CGCTTTCCCCTG | |
| U1 Terminator | ACTTTCTGGAGTTTCAAAAGTAGACTGTACGCTAA | 37 |

TABLE 5

| | Sequence (5′-3′) | SEQ ID NO |
|---|---|---|
| U7 plasmid | GGCTTAACAACAACGAAGGGGCTGTGACTGGCTGCTTTCTCAACCAA | 38 |
| sequence | TCAGCACCGAACTCATTTGCATGGGCTGAGAACAAATGTTCGCGAAC | |
| | TCTAGAAATGAATGACTTAAGTAAGTTCCTTAGAATATTATTTTTCCT | |
| | ACTGAAAGTTACCACATGCGTCGTTGTTTATACAGTAATAGGAACAA | |
| | GAAAAAAGTCACCTAAGCTCACCCTCATCAATTGTGGAGTTCCTTTA | |
| | TATCCCATCTTCTCTCCAAACACATACGCAGCGGGTCTTCGAGAAGA | |
| | CCTAGAATTTTTGGAGTAGGCTTTCTGGCTTTTTACCGGAAAGCCCCT | |
| | CTTATGATGTTTGTTGCCAATGATAGATTGTTTTCACTGTGCAAAAAT | |
| | TATGGGTAGTTTTGGTGGTCTTGATGCAGTTGTAAGCTTGGGGTATG | |
| | AAGGTTTGGGCCACGCCTGGG | |
| U7 Promoter | GGCTTAACAACAACGAAGGGGCTGTGACTGGCTGCTTTCTCAACCAA | 39 |
| | TCAGCACCGAACTCATTTGCATGGGCTGAGAACAAATGTTCGCGAAC | |
| | TCTAGAAATGAATGACTTAAGTAAGTTCCTTAGAATATTATTTTTCCT | |
| | ACTGAAAGTTACCACATGCGTCGTTGTTTATACAGTAATAGGAACAA | |
| | GAAAAAAGTCACCTAAGCTCACCCTCATCAATTGTGGAGTTCCTTTA | |
| | TATCCCATCTTCTCTCCAAACACATACGCAG | |
| Placeholder | GGGTCTTCGAGAAGACCT | 40 |
| site with 2x | | |
| opposing BbsI | | |
| type II RE sites | | |
| U7 smOPT | AGAATTTTTGGAGTAGGCTTTCTGGCTTTTTACCGGAAAGCCCCT | 41 |
| snRNA | | |
| backbone | | |
| U7 Terminator | CTTATGATGTTTGTTGCCAATGATAGATTGTTTTCACTGTGCAAAAAT | 42 |
| | TATGGGTAGTTTTGGTGGTCTTGATGCAGTTGTAAGCTTGGGGTATG | |
| | AAGGTTTGGGCCACGCCTGGG | |

### Guide Sequences

Guide sequences were designed for U1 and U7 smOPT snRNA constructs. When cloned, guide sequences provided herein replaced the “Placeholder site with 2× opposing BbsI type II RE sites” sequence. Guide sequences as described herein are oriented 5′-3′. See, Tables 1 and 2.

### Example 1—CUG Targeting—DMPK Reporter

HEK293xT cells were cultured in DMEM+10% FBS+1× penstrep medium. Dry trypsinization was performed then detached HEK cells were counted. Cells were diluted to 300K/mL (3*10⁵/mL), and plated at 300 uL per well of 48-well plate. Cells were cultured until 60%-80% confluent (overnight).

JetOPTIMUS reagents were warmed to room temperature, wherein the jetOPTIMUS mixture was made by making large master mix, wherein for each well 0.25 uL jetOPTIMUS reagent was diluted in 12.5 uL jetOPTIMUS buffer and vortexed for 2-3 seconds. The reporter plasmid mixture was then made by, for each well, adding 62.5 ng CUG960 DMPK Tet A reporter plasmid to 12.5 uL jetOPTIMUS buffer and vortexing for 2-3 seconds. A targeting plasmid mixture was made by, for each well, adding 187.5 ng of targeting plasmid to the reporter plasmid mixture above and mixing by vortexing. The jetOPTIMUS mixture is then mixed with the reporter/targeting plasmid mixture and incubated for 10 minutes at room temperature.

The incubated mixture was then dispensed onto the cells, dropping gently onto the surface of the media in each well (do not insert pipette into media). After ˜48 hours post-transfection, transfection efficiency was checked by evaluating fluorescent expression in transfection control conditions (wells transfected with a plasmid expressing a fluorescent protein). To harvest cells, media was aspirated, washed 1× with 500 uL 1×PBS, then detached/resuspended HEKs with 500 uL PBS and transfered to 1.7 mL tubes. The cells were harvested by spinning at 400×g for 5 minutes, aspirating supernatant, flash freezing in dry ice/ethanol bath, then storing at −80° C. until RNA extraction. RNA was then extracted with RNeasy (Qiagen) or preferred kit, eluting RNA using 20 uL. 6 uL of the eluted RNA was input into a reverse transcription reaction using NEB's ProtoScript II (20 uL total reaction volume).

•GAPDH primers
-Primer 1:
(SEQ ID NO: 43)
GTCTCCTCTGACTTCAACAGCG
-Primer 2:
(SEQ ID NO: 44)
ACCACCCTGTTGCTGTAGCCAA
•DMPK primers
-Primer 1:
(SEQ ID NO: 45)
TCGGAGCGGTTGTGAACT
-Primer 2:
(SEQ ID NO: 46)
GTTCGCCGTTGTTCTGTC

### Example 2—CAG Targeting—ATXN2 Reporter

HEK293xT cells were cultured in DMEM+10% FBS+1× penstrep medium. Dry trypsinization was performed then detached HEK cells were counted. Cells were diluted to 300K/mL (3*10⁵/mL), and plated at 300 uL per well of 48-well plate. Cells were cultured until 60%-80% confluent (overnight).

JetOPTIMUS reagents were warmed to room temperature, wherein the jetOPTIMUS mixture was made by making large master mix, wherein for each well 0.25 uL jetOPTIMUS reagent was diluted in 12.5 uL jetOPTIMUS buffer and vortexed for 2-3 seconds. The reporter plasmid mixture was then made by, for each well, adding 62.5 ng ATXN2 Q57 reporter plasmid to 12.5 uL jetOPTIMUS buffer and vortexing for 2-3 seconds. A targeting plasmid mixture was made by, for each well, adding 187.5 ng of targeting plasmid to the reporter plasmid mixture above and mixing by vortexing. The jetOPTIMUS mixture is then mixed with the reporter/targeting plasmid mixture and incubated for 10 minutes at room temperature.

The incubated mixture was then dispensed onto the cells, dropping gently onto the surface of the media in each well (do not insert pipette into media). After −48 hours post-transfection, transfection efficiency was checked by evaluating fluorescent expression in transfection control conditions (wells transfected with a plasmid expressing a fluorescent protein). To harvest cells, media was aspirated, washed 1× with 500 uL 1×PBS, then detached/resuspended HEKs with 500 uL PBS and transfered to 1.7 mL tubes. The cells were harvested by spinning at 400×g for 5 minutes, aspirating supernatant, flash freezing in dry ice/ethanol bath, then storing at −80° C. until RNA extraction. RNA was then extracted with RNeasy (Qiagen) or preferred kit, eluting RNA using 20 uL. 6 uL of the eluted RNA was input into a reverse transcription reaction using NEB's ProtoScript II (20 uL total reaction volume).

•GAPDH primers
-Primer 1:
(SEQ ID NO: 43)
GTCTCCTCTGACTTCAACAGCG
-Primer 2:
(SEQ ID NO: 44)
ACCACCCTGTTGCTGTAGCCAA
•ATXN2 (mCherry) primers
-Primer 1:
(SEQ ID NO: 47)
ACGGCGAGTTCATCTACAAG
-Primer 2:
(SEQ ID NO: 48)
TTCAGCCTCTGCTTGATCTC

### Example 3—Mechanistic Study (CUG/CAG)

HEK293xT cells were cultured in DMEM+10% FBS+1× penstrep medium. Dry trypsinization was performed then detached HEK cells were counted. Cells were diluted to 300K/mL (3*10⁵/mL), and plated at 300 uL per well of 48-well plate. Cells were cultured until 60%-80% confluent (overnight).

JetOPTIMUS reagents were warmed to room temperature, wherein the jetOPTIMUS mixture was made by making large master mix, wherein for each well 0.25 uL jetOPTIMUS reagent was diluted in 12.5 uL jetOPTIMUS buffer and vortexed for 2-3 seconds. The reporter plasmid mixture was then made by, for each well, adding 62.5 ng ATXN2 Q57 reporter plasmid to 12.5 uL jetOPTIMUS buffer and vortexing for 2-3 seconds. A targeting plasmid mixture was made by, for each well, adding 187.5 ng of targeting plasmid to the reporter plasmid mixture above and mixing by vortexing. The jetOPTIMUS mixture is then mixed with the reporter/targeting plasmid mixture and incubated for 10 minutes at room temperature.

The incubated mixture was then dispensed onto the cells, dropping gently onto the surface of the media in each well (do not insert pipette into media). After ˜48 hours post-transfection, transfection efficiency was checked by evaluating fluorescent expression in transfection control conditions (wells transfected with a plasmid expressing a fluorescent protein). To harvest cells, media was aspirated, washed 1× with 500 uL 1×PBS, then detached/resuspended HEKs with 500 uL PBS and transfered to 1.7 mL tubes. The cells were harvested by spinning at 400×g for 5 minutes, aspirating supernatant, flash freezing in dry ice/ethanol bath, then storing at −80° C. until RNA extraction. RNA was then extracted with RNeasy (Qiagen) or preferred kit, eluting RNA using 20 uL. 6 uL of the eluted RNA was input into a reverse transcription reaction using NEB's ProtoScript II (20 uL total reaction volume).

•GAPDH primers
-Primer 1:
(SEQ ID NO: 43)
GTCTCCTCTGACTTCAACAGCG
-Primer 2:
(SEQ ID NO: 44)
ACCACCCTGTTGCTGTAGCCAA
•DMPK primers
-Primer 1:
(SEQ ID NO: 45)
TCGGAGCGGTTGTGAACT
-Primer 2:
(SEQ ID NO: 46)
GTTCGCCGTTGTTCTGTC
•ATXN2 (mCherry) primers
-Primer 1:
(SEQ ID NO: 47)
ACGGCGAGTTCATCTACAAG
-Primer 2:
(SEQ ID NO: 48)
TTCAGCCTCTGCTTGATCTC

### Example 4—Luciferase Assay

HEK293xT cells were cultured in DMEM+10% FBS+1× penstrep medium. Dry trypsinization was performed then detached HEK cells were counted. Cells were diluted to 300K/mL (3*10⁵/mL), and plated at 300 uL per well of 48-well plate. Cells were cultured until 70%-90% confluent (overnight).

JetOPTIMUS reagents were warmed to room temperature, wherein the jetOPTIMUS mixture was made by, for each well, 0.13 uL jetOPTIMUS reagent was diluted in 6.25 uL jetOPTIMUS buffer and vortexed for 2-3 seconds. Luciferase plasmid mixtures were made by, for each well, adding 50 ng luciferase plasmid pairs to 6.25 uL jetOPTIMUS buffer and vortexing 2-3 seconds.

Luciferase plasmid pair 1 (50 ng) includes 25 ng FLuc+25 ng RLuc-MS2, and luciferase plasmid pair 2 (50 ng) includes 25 ng FLuc-MS2+25 ng RLuc. Concentrations of RNA binding protein (RBP) tethering plasmids were normalized to a shared concentration. The jetOPTIMUS mixture was then mixed with the corresponding luciferase plasmid mixture, wherein for each well, 75 ng of RBP tethering plasmid was added and mixed well. The mixture was incubated for 15 minutes at room temperature.

The incubated mixture was then dispensed onto the cells, dropping gently onto the surface of the media in each well (do not insert pipette into media). After ˜48 hours post-transfection, luminescence measurements were determined. All Dual-Glo® Luciferase Assay System reagents were thawed at room temperature (do not warm above 25° C.). The contents of one bottle of Dual-Glo® Luciferase Buffer was then transferred to one bottle of Dual-Glo® Luciferase Substrate to make Dual-Glo® Luciferase Reagent. The amount of Dual-Glo® Stop & Glo® Reagent needed for the experiment was calculated, and using a new container, the Dual-Glo® Stop & Glo® Substrate 1:100 was diluted into Dual-Glo® Stop & Glo® Buffer to make the needed volume of Dual-Glo® Stop & Glo® Reagent.

The media was then changed (replaced with 75 uL) and transfered to 96-well all white plate, wherein to each plate well, a volume of Dual-Glo® Reagent equal to the volume of culture medium was added in the well and mixed. (For 96-well plates, add 75 μl of Reagent to cells grown in 75 μl of medium.) The cells were incubated at least 10 minutes to allow for cell lysis to occur, then the firefly luminescence was measured in a luminometer.

What is claimed is:

1. A nuclear expressed antisense RNA for interfering with a target repetitive RNA, the nuclear expressed antisense RNA comprising:
(a) an oligonucleotide encoding an RNA-targeting guide comprising a sequence complementary to the target repetitive RNA; and
(b) an oligonucleotide encoding a RNA binding protein (RBP)/ribonucleoprotein (RNP)-recruiting motif.

2. The nuclear expressed antisense RNA of claim 1, wherein the RBP/RNP-recruiting motif is at the 3′ end of the RNA targeting guide.

3. The nuclear expressed antisense RNA of claim 1, wherein the RBP/RNP-recruiting motif is at the 5′ end of the RNA targeting guide.

4. The nuclear expressed antisense RNA of any one of claims 1-3, wherein the nuclear expressed antisense RNA binds to the target repetitive RNA.

5. The nuclear expressed antisense RNA of any one of claims 1-4, wherein the target repetitive RNA comprises CUG repeats, CAG repeats, or G4C2 repeats.

6. The nuclear expressed antisense RNA of any one of claims 1-5, wherein the target repetitive RNA comprises a plurality of CUG repeats.

7. The nuclear expressed antisense RNA of any one of claims 1-5, wherein the target repetitive RNA comprises a plurality of CAG repeats.

8. The nuclear expressed antisense RNA of claim 6, wherein the RNA-targeting guide comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14.

9. The nuclear expressed antisense RNA of claim 7, wherein the RNA-targeting guide comprises SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

10. The nuclear expressed antisense RNA of any one of claims 1-9, wherein the RNA-targeting guide comprises a mutation within the sequence complementary to the target repetitive RNA.

11. The nuclear expressed antisense RNA of claim 10, wherein the mutation comprises one or more mutation(s), and wherein the one or more mutation(s) comprise(s) a mismatch, deletion, insertion mutation, or any combination thereof.

12. The nuclear expressed antisense RNA of any one of claim 1-11, wherein the RBP/RNP-recruiting motif recruits an endogenous mammalian RBP/RNP.

13. The nuclear expressed antisense RNA of claim 12, wherein the endogenous mammalian RBP/RNP comprises a U snRNP component, a hnRNP component, a ribosome, a telomerase, a vault ribonucleoprotein, or an RNase P.

14. The nuclear expressed antisense RNA of any one of claims 1-13, wherein the RBP/RNP-recruiting motif comprises a U1 snRNA, a U7 smOPT snRNA, or a U7 snRNA backbone.

15. The nuclear expressed antisense RNA of claim 14, wherein the RBP/RNP-recruiting motif comprises a U1 snRNA, wherein the U1 snRNA comprises SEQ ID NO: 29, SEQ ID NO: 30, SEQ ID NO: 31, or SEQ ID NO: 32.

16. The nuclear expressed antisense RNA of any one of claims 1-15, wherein the nuclear expressed antisense RNA is delivered into a cell.

17. The nuclear expressed antisense RNA of claim 16, wherein the cell is a mammalian cell.

18. The nuclear expressed antisense RNA of claim 16, wherein the nuclear expressed antisense RNA is delivered by transfection, transduction, mechanical delivery, lipid, or transporter protein.

19. The nuclear expressed antisense RNA of claim 18, wherein the nuclear expressed antisense RNA is delivered by a transfection reagent.

20. The nuclear expressed antisense RNA of any one of claims 1-19, wherein the nuclear expressed antisense RNA is comprised in a vector.

21. The nuclear expressed antisense RNA of claim 20, wherein the vector is a viral vector.

22. The nuclear expressed antisense RNA of claim 21, wherein the viral vector is an adeno-associated viral vector (AAV), lentiviral vector, or an adenoviral vector.

23. The nuclear expressed antisense RNA of any one of claims 1-22, wherein the nuclear expressed antisense RNA further comprises a Polymerase II promoter or a Polymerase III promoter sequence.

24. The nuclear expressed antisense RNA of claim 23, wherein the nuclear expressed antisense RNA comprises a U1, U6, or U7 promoter.

25. A vector comprising a nucleic acid encoding a nuclear expressed antisense RNA comprising (i) an RNA-targeting guide comprising a sequence complementary to a target repetitive RNA, and (ii) a RBP/RNP-recruiting motif.

26. The vector of claim 25, wherein the RBP/RNP-recruiting motif is at the 3′ end of the RNA-targeting guide.

27. The vector of claim 25, wherein the RBP/RNP-recruiting motif is at the 5′ end of the RNA-targeting guide.

28. The vector of any one of claims 25-27, wherein the RNA-targeting guide binds to the target repetitive RNA.

29. The vector of any one of claims 25-28, wherein the target repetitive RNA comprises CUG repeats, CAG repeats, or G4C2 repeats.

30. The vector of claim 29, wherein the target repetitive RNA comprises a plurality of CUG repeats.

31. The vector of claim 29, wherein the target repetitive RNA comprises a plurality of CAG repeats.

32. The vector of claim 30, wherein the RNA-targeting guide comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14.

33. The vector of claim 31, wherein the RNA-targeting guide comprises SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

34. The vector of any one of claims 25-33, wherein the RNA-targeting guide comprises a mutation within the sequence complementary to the target repetitive RNA.

35. The vector of claim 34, wherein the mutation comprises a mismatch, deletion, or insertion mutation.

36. The vector of any one of claim 25-35, wherein the RBP/RNP-recruiting motif recruits an endogenous mammalian RBP/RNP.

37. The vector of claim 36, wherein the endogenous mammalian RBP/RNP comprises a U snRNP component, a hnRNP component, a ribosome, a telomerase, a vault ribonucleoprotein, or an RNase P.

38. The vector of any one of claims 25-37, wherein the RBP/RNP-recruiting motif comprises a U1 snRNA, a U7 smOPT snRNA, or a U7 snRNA backbone.

39. The vector of claim 38, wherein the RBP/RNP-recruiting motif comprises a U1 snRNA, wherein the U1 snRNA comprises SEQ ID NO: 29, SEQ ID NO: 30, SEQ ID NO: 31, or SEQ ID NO: 32.

40. The vector of any one of claims 25-39, wherein the vector is delivered into a cell.

41. The vector of claim 40, wherein the cell is a mammalian cell.

42. The vector of claim 40, wherein the vector is delivered by transfection, transduction, mechanical delivery, lipid, or transporter protein.

43. The vector of claim 42, wherein the vector is delivered by a transfection reagent.

44. The vector of any one of claims 25-43, wherein the vector is a viral vector.

45. The vector of claim 44, wherein the viral vector is an adeno-associated viral vector (AAV), lentiviral vector, or an adenoviral vector.

46. The vector of any one of claims 25-45, wherein the vector further comprises a Polymerase II promoter or a Polymerase III promoter sequence.

47. The vector of claim 46, wherein the vector comprises a U1, U6, or U7 promoter.

48. A cell comprising a nuclear expressed antisense RNA of any one of claims 1-24 or a vector of any one of claims 25-47.

49. A pharmaceutical composition comprising the cell of claim 48 and a pharmaceutically acceptable carrier.

50. A method of treating a disease associated with expansions of a target repetitive RNA in a subject, the method comprising administering to the subject a therapeutically effective amount of the pharmaceutical composition of claim 49.

51. The method of claim 50, wherein the disease is myotonic dystrophy, amyotrophic lateral sclerosis, frontotemporal dementia, Huntington disease, Friedreich ataxia, oculopharyngeal muscular dystrophy, spinocerebellar ataxia, or spinal and bulbar muscular atrophy.

52. A method of preventing nuclear accumulation and/or protein translation of a target repetitive RNA in a cell, the method comprising delivering a nuclear expressed antisense RNA of any one of claims 1-24 or a vector of any one of claims 25-47.

53. The method of claim 52, wherein the cell is a mammalian cell.

54. The method of claim 53, wherein the cell is a human cell.


SEQUENCE LISTING (48 sequences)

SEQ ID NO: 1 - DNA, 25 bp, synthetic construct
  source 1..25: mol_type = other DNA
gatggaatgtccttaactctcgcac

SEQ ID NO: 2 - DNA, 15 bp, synthetic construct
  source 1..15: mol_type = other DNA
cagcagcagcagcag

SEQ ID NO: 3 - DNA, 15 bp, synthetic construct
  source 1..15: mol_type = other DNA
agcagcagcagcagc

SEQ ID NO: 4 - DNA, 15 bp, synthetic construct
  source 1..15: mol_type = other DNA
gcagcagcagcagca

SEQ ID NO: 5 - DNA, 20 bp, synthetic construct
  source 1..20: mol_type = other DNA
agcagcagcagcagcagcag

SEQ ID NO: 6 - DNA, 20 bp, synthetic construct
  source 1..20: mol_type = other DNA
gcagcagcagcagcagcagc

SEQ ID NO: 7 - DNA, 20 bp, synthetic construct
  source 1..20: mol_type = other DNA
cagcagcagcagcagcagca

SEQ ID NO: 8 - DNA, 25 bp, synthetic construct
  source 1..25: mol_type = other DNA
gatggaatgtccttaactctcgcac

SEQ ID NO: 9 - DNA, 15 bp, synthetic construct
  source 1..15: mol_type = other DNA
cagcagcagcagcag

SEQ ID NO: 10 - DNA, 15 bp, synthetic construct
  source 1..15: mol_type = other DNA
agcagcagcagcagc

SEQ ID NO: 11 - DNA, 15 bp, synthetic construct
  source 1..15: mol_type = other DNA
gcagcagcagcagca

SEQ ID NO: 12 - DNA, 20 bp, synthetic construct
  source 1..20: mol_type = other DNA
agcagcagcagcagcagcag

SEQ ID NO: 13 - DNA, 20 bp, synthetic construct
  source 1..20: mol_type = other DNA
gcagcagcagcagcagcagc

SEQ ID NO: 14 - DNA, 20 bp, synthetic construct
  source 1..20: mol_type = other DNA
cagcagcagcagcagcagca

SEQ ID NO: 15 - DNA, 25 bp, synthetic construct
  source 1..25: mol_type = other DNA
gatggaatgtccttaactctcgcac

SEQ ID NO: 16 - DNA, 15 bp, synthetic construct
  source 1..15: mol_type = other DNA
ctgctgctgctgctg

SEQ ID NO: 17 - DNA, 15 bp, synthetic construct
  source 1..15: mol_type = other DNA
tgctgctgctgctgc

SEQ ID NO: 18 - DNA, 15 bp, synthetic construct
  source 1..15: mol_type = other DNA
gctgctgctgctgct

SEQ ID NO: 19 - DNA, 20 bp, synthetic construct
  source 1..20: mol_type = other DNA
tgctgctgctgctgctgctg

SEQ ID NO: 20 - DNA, 20 bp, synthetic construct
  source 1..20: mol_type = other DNA
gctgctgctgctgctgctgc

SEQ ID NO: 21 - DNA, 20 bp, synthetic construct
  source 1..20: mol_type = other DNA
ctgctgctgctgctgctgct

SEQ ID NO: 22 - DNA, 25 bp, synthetic construct
  source 1..25: mol_type = other DNA
gatggaatgtccttaactctcgcac

SEQ ID NO: 23 - DNA, 15 bp, synthetic construct
  source 1..15: mol_type = other DNA
ctgctgctgctgctg

SEQ ID NO: 24 - DNA, 15 bp, synthetic construct
  source 1..15: mol_type = other DNA
tgctgctgctgctgc

SEQ ID NO: 25 - DNA, 15 bp, synthetic construct
  source 1..15: mol_type = other DNA
gctgctgctgctgct

SEQ ID NO: 26 - DNA, 20 bp, synthetic construct
  source 1..20: mol_type = other DNA
tgctgctgctgctgctgctg

SEQ ID NO: 27 - DNA, 20 bp, synthetic construct
  source 1..20: mol_type = other DNA
gctgctgctgctgctgctgc

SEQ ID NO: 28 - DNA, 20 bp, synthetic construct
  source 1..20: mol_type = other DNA
ctgctgctgctgctgctgct

SEQ ID NO: 29 - DNA, 16 bp, synthetic construct
  source 1..16: mol_type = other DNA
ataatttgtggtagtg

SEQ ID NO: 30 - DNA, 26 bp, synthetic construct
  source 1..26: mol_type = other DNA
ggggactgcgttcgcgctttcccctg

SEQ ID NO: 31 - DNA, 44 bp, synthetic construct
  source 1..44: mol_type = other DNA
cagggcgaggcttatccattgcactccggatgtgctgacccctg

SEQ ID NO: 32 - DNA, 33 bp, synthetic construct
  source 1..33: mol_type = other DNA
gggagataccatgatcacgaaggtggttttccc

SEQ ID NO: 33 - DNA, 609 bp, synthetic construct
  source 1..609: mol_type = other DNA
taacacaggctaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcccaagatctcagggtcttcgagaagacctgcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctgactttctggagtttcaaaagtagactgtacgctaa

SEQ ID NO: 34 - DNA, 402 bp, synthetic construct
  source 1..402: mol_type = other DNA
taacacaggctaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcccaagatctc

SEQ ID NO: 35 - DNA, 18 bp, synthetic construct
  source 1..18: mol_type = other DNA
gggtcttcgagaagacct

SEQ ID NO: 36 - DNA, 153 bp, synthetic construct
  source 1..153: mol_type = other DNA
gcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctg

SEQ ID NO: 37 - DNA, 35 bp, synthetic construct
  source 1..35: mol_type = other DNA
actttctggagtttcaaaagtagactgtacgctaa

SEQ ID NO: 38 - DNA, 447 bp, synthetic construct
  source 1..447: mol_type = other DNA
ggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcagcgggtcttcgagaagacctagaatttttggagtaggctttctggctttttaccggaaagcccctcttatgatgtttgttgccaatgatagattgttttcactgtgcaaaaattatgggtagttttggtggtcttgatgcagttgtaagcttggggtatgaaggtttgggccacgcctggg

SEQ ID NO: 39 - DNA, 267 bp, synthetic construct
  source 1..267: mol_type = other DNA
ggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcag

SEQ ID NO: 40 - DNA, 18 bp, synthetic construct
  source 1..18: mol_type = other DNA
gggtcttcgagaagacct

SEQ ID NO: 41 - DNA, 45 bp, synthetic construct
  source 1..45: mol_type = other DNA
agaatttttggagtaggctttctggctttttaccggaaagcccct

SEQ ID NO: 42 - DNA, 116 bp, synthetic construct
  source 1..116: mol_type = other DNA
cttatgatgtttgttgccaatgatagattgttttcactgtgcaaaaattatgggtagttttggtggtcttgatgcagttgtaagcttggggtatgaaggtttgggccacgcctggg

SEQ ID NO: 43 - DNA, 22 bp, synthetic construct
  source 1..22: mol_type = other DNA
gtctcctctgacttcaacagcg

SEQ ID NO: 44 - DNA, 22 bp, synthetic construct
  source 1..22: mol_type = other DNA
accaccctgttgctgtagccaa

SEQ ID NO: 45 - DNA, 18 bp, synthetic construct
  source 1..18: mol_type = other DNA
tcggagcggttgtgaact

SEQ ID NO: 46 - DNA, 18 bp, synthetic construct
  source 1..18: mol_type = other DNA
gttcgccgttgttctgtc

SEQ ID NO: 47 - DNA, 20 bp, synthetic construct
  source 1..20: mol_type = other DNA
acggcgagttcatctacaag

SEQ ID NO: 48 - DNA, 20 bp, synthetic construct
  source 1..20: mol_type = other DNA
ttcagcctctgcttgatctc